(19) United States
(12) Patent Application Publication
(10) Pub. No.: US 2025/0032336 A1
(43) Pub. Date: Jan. 30, 2025

(54) A HOOD

(71) Applicant: The University of Bristol, Bristol (GB)

(72) Inventors: Mojtaba Dorri, Bristol (GB); Alberto Gambaruto, Bristol (GB)

(21) Appl. No.: 18/580,111

(22) PCT Filed: Jul. 15, 2022
(86) PCT No.: PCT/GB2022/051839

§ 371 Date: Jan. 17, 2024

(30) Foreign Application Priority Data
Jul. 19, 2021 (GB) 2110319.7

(51) Int. Cl. A61G 10/00; A61M 1/00

(52) U.S. Cl. CPC A61G 10/005; A61M 1/80

(57) ABSTRACT

A hood comprising: a barrier defining an interior space for receiving the head of a patient and an opening through which the head may enter and leave the interior space, the barrier being arranged to prevent the transmission of aerosol particles through the barrier; a suction port arranged to remove air from the interior space of the hood; an access hole within the barrier, arranged to allow a person external to the barrier to access the interior space; and an air jet arranged to propel air across the access hole in order to inhibit the transmission of aerosols through the access hole.

### BACKGROUND

During medical procedures, there is a danger of pathogens being transferred from the patient to the medical practitioner and the clinic environment. While the wearing of masks and suchlike may reduce the prospect of the transfer of pathogens, this may not always be possible. In particular, for dental procedures it may be necessary that a medical practitioner must be able to access the airways of a patient, such as the mouth, nose and throat, and so a mouth and nose covering such as a mask may not be used.

Further, even in cases where the medical practitioner may be protected by their own mask, there may be the prospect that airborne pathogens may be suspended within the room in which the medical procedure takes place and so may be inhaled by a subsequent patient who may have a later medical procedure in the same room.

While generic coverings and extraction and ventilation systems may reduce the amount of airborne pathogens, certain medical procedures, such as drilling teeth, may produce high momentum particles that may escape from conventional air circulation devices and may remain in the room.

Overall, therefore, there is a need for a hood that may inhibit the spread of airborne particles from a medical procedure, while allowing the medical practitioner to carry out the medical procedure.

### SUMMARY OF THE INVENTION

According to a first aspect of the invention, there is provided a hood comprising: a barrier defining an interior space for receiving the head of a patient and an opening through which the head may enter and leave the interior space, the barrier being arranged to prevent the transmission of aerosol particles through the barrier; a suction port arranged to remove air from the interior space of the hood; an access hole within the barrier, arranged to allow a person external to the barrier to access the interior space; and an air jet arranged to propel air across the access hole in order to inhibit the transmission of aerosols through the access hole.

With such a hood, the access hole may allow the medical practitioner to access the interior of the hood and the medical practitioner may therefore carry out a medical procedure as usual. Meanwhile, the air jet may prevent the transmission of airborne particles, such as pathogens, through the access hole by providing a high momentum stream of gas in order to divert the path of any pathogens or other airborne particles that were previously directed towards the access hole, such that to the airborne particles remain within the hood. The airborne pathogens may then subsequently be extracted via the suction port.

The barrier of the hood may be made from a material that is substantially impermeable to air, and may be transparent in order to allow the medical practitioner to see the patient. The inner surface of the barrier may be treated with an anti-fog coating and/or the outer surface of the barrier may be treated with an anti-glare coating.

Once an air particle is prevented from leaving the hood via the access hole, the air particle may be removed from the hood via the suction port, which may be coupled to a suction means. The suction port may therefore be suitable for coupling to or arranged to couple to a suction means.

In cases where a hood may be provided only with a suction port, and without an air jet, there may be two drawbacks. Firstly, the suction port may be arranged a distance from the access hole and so any high momentum particles travelling towards the access hole may be insufficiently deflected by the bulk air flow and may still leave via the access hole. Further, the suction through the suction port may result in a low pressure within the hood, which may reduce the efficiency of a pump coupled to the suction port and may be uncomfortable for a patient. By providing an air jet, which may propel air into the hood, the pressure within the hood may be maintained at substantially atmospheric pressure.

The hood may comprise a plurality of air jets arranged to drive air across the access hole in order to inhibit the transition of aerosols through the access hole. By providing a plurality of air jets, the transmission of aerosols or other airborne particles through the access hole may be further reduced, and an air curtain may be formed, which may envelope or surround the hands or wrists of a medical practitioner that are within the access hole. Overall, transmission of aerosols through the access hole may be reduced.

The plurality of air jets may be arranged parallel and may be arranged in a row extending perpendicularly to the direction of flow of the jets. This may thereby provide an air curtain that may be substantially uniform across the access hole.

The air jets may be distributed around a boundary of the access hole and may be directed across the access hole. The prevention of aerosols being transmitted through the access hole may therefore be reduced further.

The air jets may be powered via a common manifold and/or may be powered by a common source. This may provide a more compact and simpler system for powering the air jets.

The air jet or, where there are plural air jets, the air jets, may be directed toward the suction port. This may provide an overall flow regime within the hood toward the suction port and may thereby improve the extraction of airborne particles and reduce the recirculation of airborne particles. Overall, the concentration of airborne particles within the hood, and therefore the prospect of the escape of airborne particles, may be reduced.

The air jets may be holes in the barrier arranged to permit the flow of air therethrough when a low pressure is created in the interior space by the removal of air via the suction port. In this way, the transmission of aerosols through the access port may be inhibited without the requirement to provide a dedicated air pumping means for the air jets, and the air jets may effectively be powered by the suction port.

The hood may further comprise a first flap extending at least partially across the access hole in a first direction. The flap may inhibit the passage of aerosols through the access holes by effectively reducing the cross sectional area of the access hole, without impeding a medical practitioner accessing the interior space.

The hood may further comprise a second flap extending at least partially across the access hole in a second direction different from the first direction and arranged to overlap the first flap. Optionally, the second direction may be opposite to the first direction. Alternatively, the hood may comprise a plurality of flaps arranged to overlap each other circumferentially around the access hole, this may be in the manner of a camera aperture. The provision of a second or further flap may further inhibit the transmission of aerosols through the access hole, and may improve a seal about the hand of a medical professional extending through the access hole.

The first and second flaps may have edges that are shaped as sinusoids in the regions where the flaps overlap, and the sinusoids may be 180° out of phase. This may provide a flap edge which may more closely envelope the wrist of a medical practitioner who is using the access hole, consequently reducing the transmission of aerosols through the access hole.

The hood may further comprise a first skirt extending from the barrier and arranged to at least partially obstruct the opening. The first skirt may be arranged to go around a dentist's chair upon which the hood may be arranged. The first skirt may therefore be formed from an elastic material and may be arranged over the opening as a diaphragm connected only around a portion of its periphery.

The first skirt may have a quarter-spheroidal or quarter-ellipsoidal shape. This may allow the first skirt to fit around a chair upon which the patient may be seated and around the patient's torso.

The hood may further comprise a second skirt extending from the barrier and having an edge distal from the barrier, the edge having a concave shape. The edge of the barrier having a concave shape may sit on a patient's chest and may reduce the transmission of aerosols through the opening of the hood.

The barrier may be hemi-spheroidal or hemi-ellipsoidal. This may provide a well-shaped interior space for accommodating the head of a patient.

The hood may also comprise lighting arranged inside the barrier. The lighting may be arranged inside the barrier and directed toward the interior of the barrier, such that it may illuminate the face or mouth of a patient when the patient is in situ.

According to a second aspect of the invention, there is provided an airflow system comprising: a hood according to the first aspect of the invention; and a suction pump coupled to the suction port arranged to remove air from the interior space. The air flow system may be arranged to provide air flow through the hood such that airborne particles within the hood are prevented from leaving via the access holes by the air jet(s) and may be removed from the hood via the suction port.

The suction pump may be arranged to extract air from the hood at a rate of at least 70 Litres per minute, preferably at least 1500 litres per minute. The suction pump may be arranged to extract air from the hood at a rate such that the volume of air extracted each second is greater than or equal to the internal volume of the barrier.

The air flow system may further comprise a jet pump coupled to the air jet(s) and arranged to provide air to the jets at a pressure above atmospheric pressure.

The suction pump and the jet pump may be the same pump. This may allow the airflow system to be operated as a circuit using only a single pump, thereby providing a more simple system. Air removed from the interior space via the suction port may be decontaminated and reinjected via the air jets.

The airflow system may further comprise an air decontamination device arranged to sanitise the air passing through the air flow system. The air decontamination device may comprise an air filter and/or a UV sterilisation device.

### BRIEF DESCRIPTION OF THE DRAWINGS

In order to provide a more complete understanding of the disclosure, the following drawings are provided, by way of example only, in which:

FIG. 1 shows a first hood according to the invention;

FIG. 2 shows the first hood and a patient situated within the hood;

FIG. 3 shows a second hood according to the invention;

FIG. 4 shows a third hood according to the invention;

FIG. 5 shows a fourth hood according to the invention;

FIG. 6 shows a fifth hood according to the invention;

FIG. 7 shows a sixth hood according to the invention;

FIG. 8 shows a seventh hood according to the invention;

FIGS. 9 and 10 show an eighth hood according to the invention in profile and front views respectively;

FIGS. 11 and 12 show a ninth hood according to the invention in profile and front views respectively;

FIG. 13 shows a tenth hood according to the invention;

FIG. 14 shows an air flow system according to the invention; and

FIG. 15 shows a further flap arrangement for use with a hood according to the invention.

### DETAILED DESCRIPTION

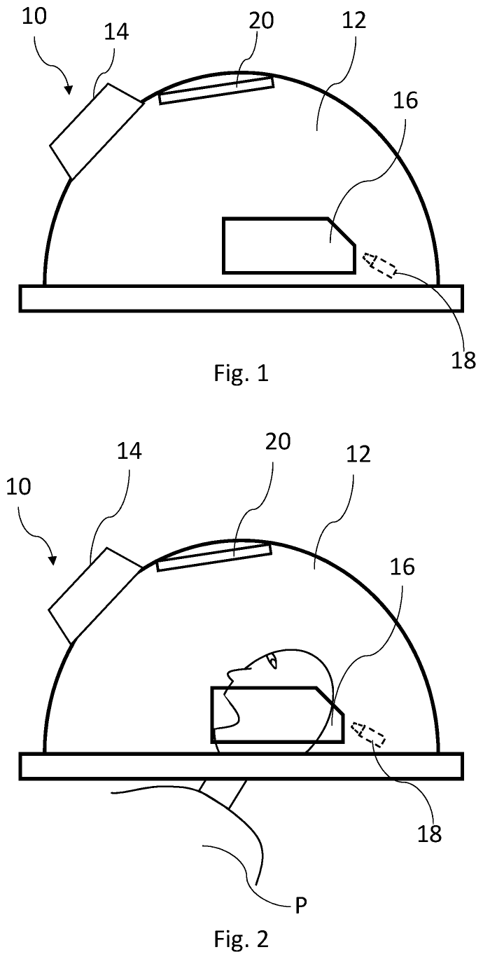

FIG. 1 shows a hood 10 having a hemispherical barrier 12, the barrier defining an interior space for receiving a patient's head and having an opening at a lower side thereof. It will be understood that the opening may be circular or elliptical, although other shapes of both the barrier 12 and of the opening are possible.

The barrier 12 has a suction port 14 arranged in it for coupling to a suction device, such as a suction hose or vacuum pump. The suction port 14 is open to the internal space of the barrier 12 so that it may be used to extract air and other particles from the interior space of the barrier 12. The barrier 12 also has at least one, and preferably two, access holes 16, formed as a void through the barrier 12 such that there is open communication between the interior of the barrier 12 and the external environment. The barrier 12 may have two access holes 16 arranged symmetrically about a plane perpendicular to the opening and passing through the suction port 14.

The access hole 16 may be sized such that a medical practitioner may insert his or her hand through the access hole 16. The access hole 16 may therefore have a smallest dimension across it of greater than 15 centimetres and less than 35 centimetres and a longest dimension across it of greater than 30 centimetres and less than 50 centimetres. Preferably, the access hole may be a rectangle having a width of 40 centimetres and a height of 20 centimetres. This may provide a hole having a sufficient size to allow access to the patient, while being sufficiently small that an excessive amount of transmission of aerosols between the interior of the barrier and the exterior of the barrier 12 is not allowed. The access hole 16 may also have any shape. While the access hole 16 is shown as a pentagon, it may be a square, rectangle, trapezium, circle, ellipse or any other shape.

The hood 10 also has an air jet 18. The air jet 18 is directed across the access hole 16 in order to induce an air flow over the access hole 16 which may prevent aerosols from passing through the access hole 16. The air jet may be powered by an air pump (not shown). The air jet 18 is arranged on an opposite side of the access hole 16 from the suction port 14, so that the air jet 18 is directed across the access hole 16 and toward the suction port 14.

The air jet 18 may be arranged to direct air substantially parallel or tangential to the barrier 12. In some cases, the air jet 18 may be arranged to create a vortical airflow around the interior volume, the air flow travelling circumferentially around the inner surface of the barrier 18. In this case, the air jet 18 may be located further from the access hole 16.

Further, the hood 10 has a light 20 arranged inside the barrier and directed toward the patient to illuminate the mouth and face of the patient.

FIG. 2 shows how a patient P may be positioned within the hood 10. The patient P may be arranged to face the suction port 14, such that the patient's mouth or airway faces the suction port 14. The hood 10 may be provided with a head support or head rest in order to hold a patient's head in this arrangement. Alternatively, the hood 10 may be arranged to be fixed to a chair having a headrest. The access hole(s) 16 may be arranged behind the patient P's head in order to allow a dentist to access the patient's mouth in the usual way. In this way, a dentist or dental assistant carrying out a dental procedure may be substantially unencumbered by the hood 12.

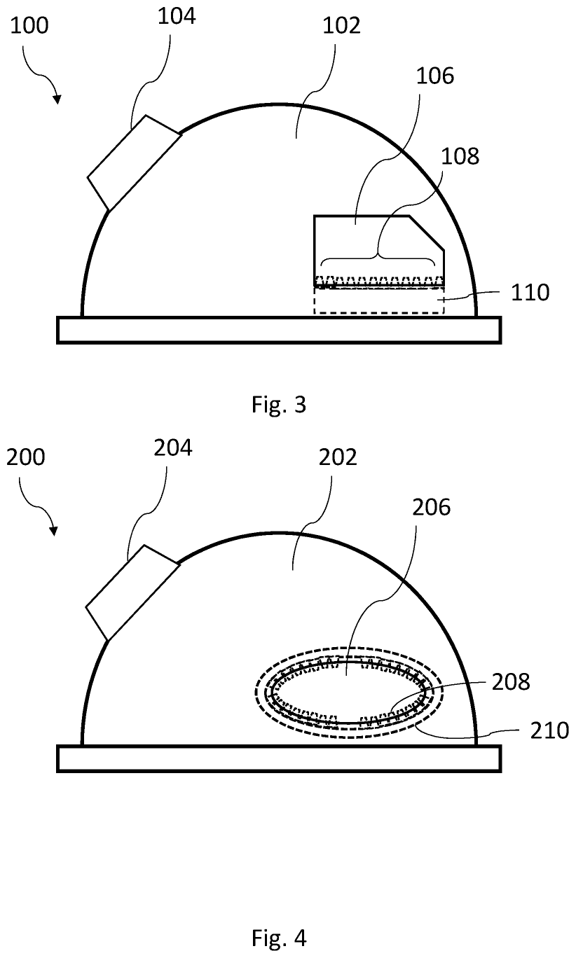

FIG. 3 shows a second hood 100, with a barrier 102, suction port 104 and access hole 106 substantially similar to those described above with reference to FIGS. 1 and 2. However, the hood 100 comprises a plurality of air jets 108, which are arranged parallel and in a row perpendicular to the direction in which the air which will be expelled from the jets is directed. The air jets are all arranged to expel air vertically and are arranged in a horizontally extending row. The air jets 108 may thereby provide a relatively uniform air curtain across the access hole 106. Further, the air jets are arranged such that they point approximately towards the suction port 104 so that there is a general bulk flow of air within the barrier 102 towards the suction port 104. The air jets 108 are powered via a single manifold 110, which means that a fewer number of parts is required for constructing the hood 100 and there may be a single connection to a high pressure air supply, improving the ease of assembly of the hood 100 within an air circulation system.

FIG. 4 shows a third hood 200 having a barrier 202 and suction port 204, which are substantially similar to those described above. However, the access hole 206 is elliptical. The choice of shape for the access hole may be selected by a practitioner depending on personal preference or may be selected according to the type of procedure being carried out. Further, the hood 200 has a plurality of air jets 208 which are arranged around the circumference of the access hole 206, each of the air jets 208 pointing across the access hole 206 such that airborne particles in the region of the access hole 206 may be deflected by the air jets 208 and may subsequently be removed via the suction port 204. The prospects of the air jets 208 being blocked by a hand extending through the access hole 206 such that a region of the access hole 206 may have no high-velocity air flowing across it may also be reduced.

As was described above with reference to FIG. 3, the plurality of air jets 208 may all be powered via a single manifold 210.

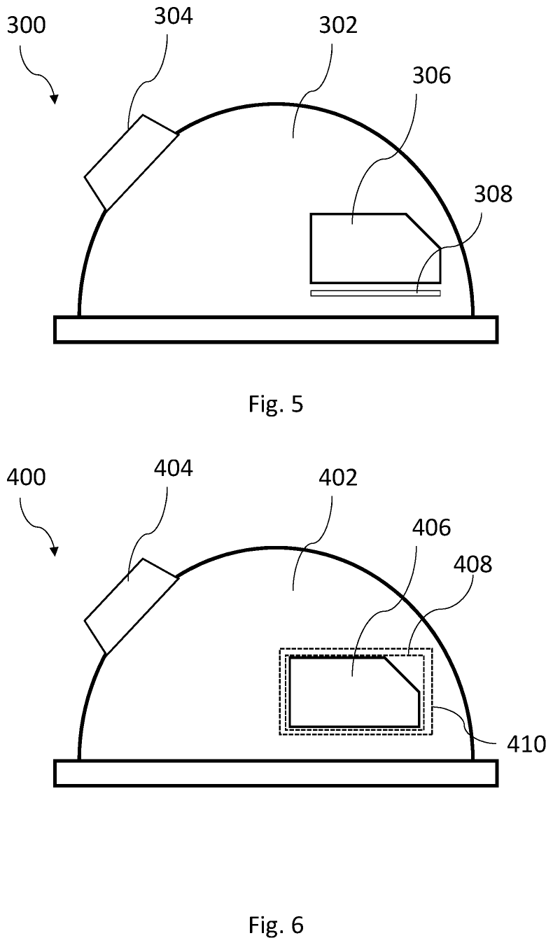

FIG. 5 shows a further hood 300 having a barrier 302, a suction port 304 and an access hole 306 substantially similar to those described above. The hood 300 further has an air jet 308 formed as a hole or slit through the barrier 302. In particular, the cross section of the air jet hole 308 may be smaller than that of the access hole 306. The hole 308 has a small cross section, such that the low pressure within the barrier 302 which may be developed by suction applied to the suction port 304 may induce high velocity air flow through the hole 308 and may provide an air jet sufficient to deflect airborne particles in the region of the access hole 306 and may thereby inhibit transmission of the particles through the access hole 306.

The hood 400 shown in FIG. 6 has an access hole 406, a barrier 402 and a suction port 404 substantially similar to those described above. However, the air jets 408 are formed as holes within a manifold 410 that surrounds the access hole 406. While the above-described air jets may be formed as convergent nozzles extending from a manifold, the air jets may alternatively be formed as a pipe having holes arranged in a row. This may be a more simple and cheaper alternative to the convergent nozzles more commonly used for providing air jets.

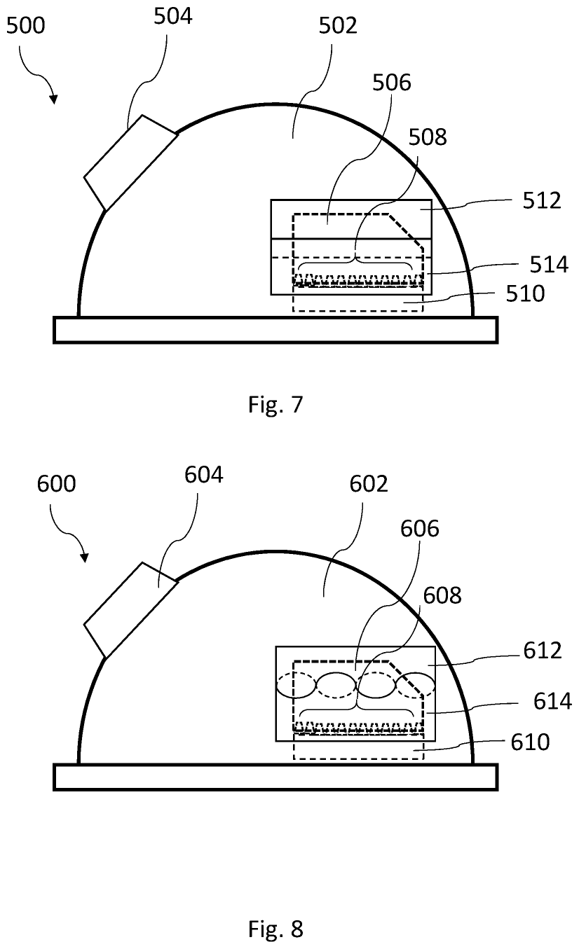

FIGS. 7 and 8 show hoods 500, 600 having barriers 502, 602, suction ports 504, 604, access holes 506, 606, air jets 508, 608 and manifolds 510, 610 substantially similar to those described above. However, the hoods 500, 600 also have flaps 512, 514, 612, 614.

Looking firstly to the hood 500 of FIG. 7, the hood 500 has two substantially rectangular flaps 512, 514. The hood has a first flap 512, which is fixed or coupled to the barrier 502 at a top edge of the flap 512 and extends downwardly therefrom. The second flap 514 is attached at a lower edge of the flap 514 and extends upwardly therefrom, such that the first and second flap 512, 514 overlap in a region over the access hole 506. The flaps may be flexible such that the medical practitioner may bend them in order to extend his or her hand through the access hole 506.

The hood 600 shown in FIG. 8 has flaps substantially similar to those described with reference to FIG. 7, except that the flaps 612, 614 of the hood 600 have edges with are sinusoidal in shape. The edges of the flaps 612, 614, opposite the edges of the flaps which are coupled to the barrier 602 have a curved shape and may be sinusoidally shaped, with the edges of the two flaps exhibiting sinusoids which are 180° out of phase, such that the flaps overlap in a region over the access hole 606. The curves of the edges of the flaps 612, 624 may cooperate, when the flaps are bent, to provide a curved hole therebetween, allowing the flaps to seal tightly about the wrist or forearm of a medical practitioner.

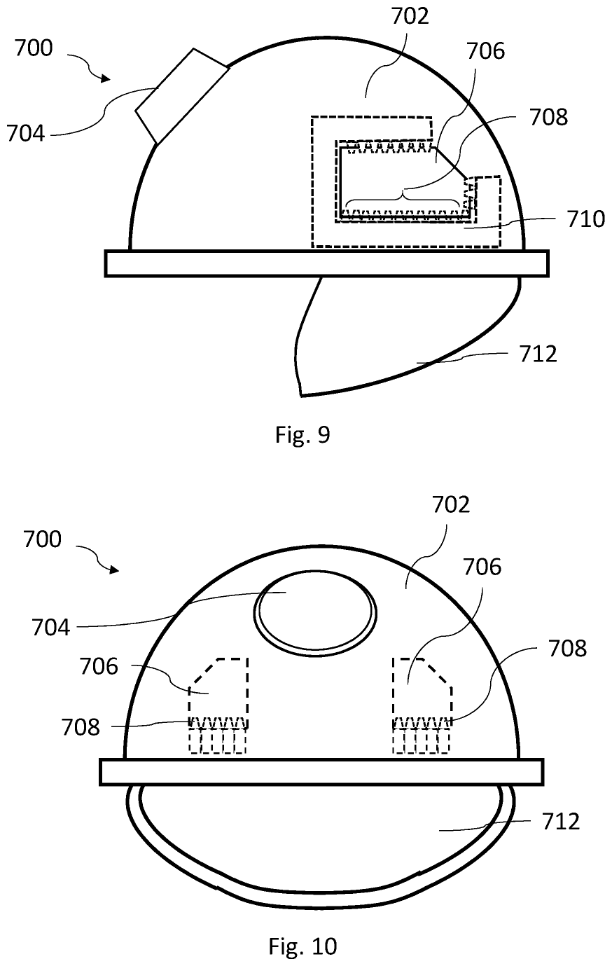

FIGS. 9 and 10 show side and front views of a hood 700 having a barrier 702, suction port 704, access port 706, air jets 708 and a manifold 710 substantially similar to those described above, with the exception that the manifold 710 may not necessarily extend about more than one side of the access hole 706 but not entirely about the periphery of the access hole 706. This may improve visibility and access to the patient while maintaining a good flow over the access hole.

The hood 700 further comprises a skirt 712 extending from the opening at the lower edge of the barrier 702 and may be quarter spherical or quarter ellipsoidal in shape and may be arranged to receive a headrest of a dentist's chair, such that the hood may seal around the chair and may have reduced leakage of airborne particles therefrom.

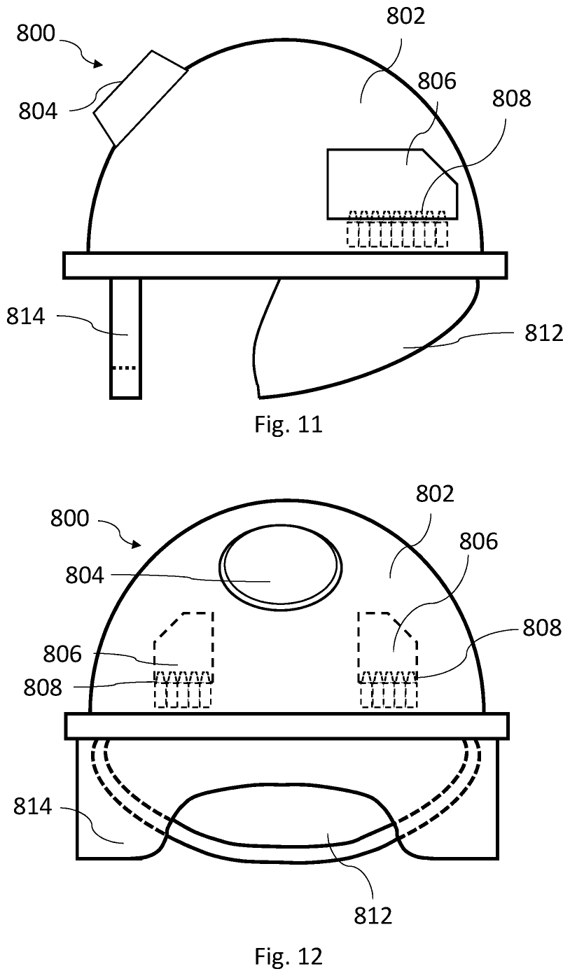

The hood 800, shown in FIGS. 11 and 12, differs from the hood 700 in that the hood 800 further comprises a second skirt 814. The second skirt is substantially planar and a lower end of the skirt 814, i.e. an end of the skirt 814 opposite to the end of the skirt at which it is coupled to the barrier 802, has a concave shape. In this way, the skirt 814 may lie comfortably on the torso of a patient and may provide a good seal to the torso of the patient, further decreasing the amount of leakage of airborne pathogens that may occur via the opening.

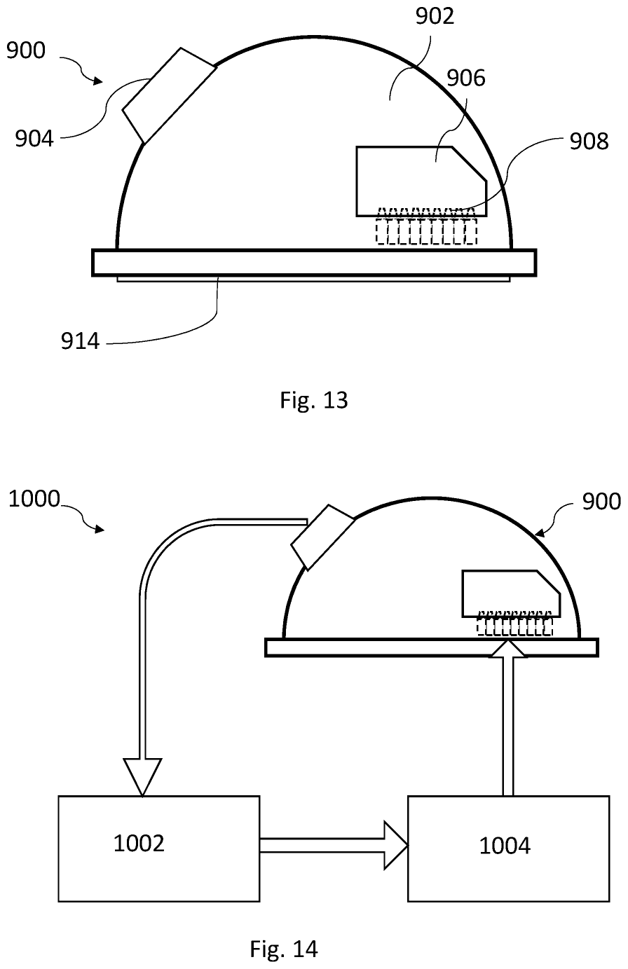

FIG. 13 shows a further hood 900 having a barrier 902, suction port 904, access hole 906 and air jets 908 substantially similar to those described above. The hood 900 further comprises a skirt 914 that is formed as an elastic diaphragm and is fixed to the barrier 902 of the hood 900 around a portion of its circumference. A portion of the circumference of the skirt 914 is not fixed to the barrier 902 and is separable from the barrier, such that the skirt 914 may be stretched in order to allow a patient to access the interior space of the barrier 902.

Further optional features that may be incorporated with any of the above-described hoods include a mirror and a magnifying lens. The mirror may be arranged in the eyeline of the patient, i.e. adjacent to the suction port, facing inwardly, or as an annular mirror surrounding the suction port, and may help to calm claustrophobic patients. The magnifying lens may be arranged in the eyeline of the dentist and so may by in an upper part of the barrier, and may be directed toward the mouth of the patent. The magnifying lens may therefore assist a dentist by improving their vision of the patient's mouth.

FIG. 14 schematically shows an air flow system 1000 incorporating the hood 900. The air flow system further comprises a pump 1002 coupled to the suction port and arranged to provide a low pressure to the suction port in order to remove air from the interior space via the suction port. The pump 1002 provide the air at a high pressure to the air jets via a decontamination device 1004.

The decontamination device 1004 may be a UV sterilisation device arranged to kill any airborne pathogens or may be an air filter, such as a HEPA filter, arranged to filter out any undesirable airborne particles.

While the air flow system 1000 of FIG. 14 is shown as a circuit, the system may be configured such that a dedicated air jet pump is arranged to power the air jets, taking in air from the atmosphere, and a separate suction pump may extract air from the hood via the suction port and may expel the air to an external environment.

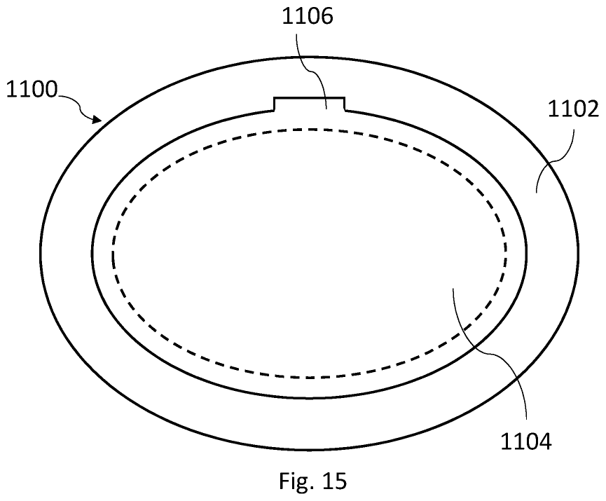

FIG. 15 shows a further flap arrangement 1100, which may be combined with any aforementioned hood. The flap arrangement 1100 has a first annular flap 1102, which is formed of a relatively elastic material. This may provide a good seal around the wrist or forearm of a medical practitioner. A second flap 1104 is formed of a relatively less elastic material and is arranged to cover a hole in the annular first flap 1102.

The second flap 1104 is hingedly coupled to the first flap 1102 at a hinge portion 1106. In use, the second flap 1104 may cover the hole in the first flap 1102 when the access hole is not in use, and may be moved by the medical practitioner when access via the access hole is required. The second flap may be arranged to move inwardly, i.e. toward the patient. The first and second flaps 1102, 1104 may also overlap in an annular overlap region. While the flaps shown are elliptical, it will be understood that their shape may vary and they may be square, rectangular, circular or any other shape.

The different parts of the aforementioned hoods may be releasably coupled. In particular, the skirts, the flaps and the air jets and manifolds may be releasably coupled to the barrier. The coupling may be made by Velcro or by other means and the releasable coupling may allow the different parts to be separated and cleaned.

1. A hood comprising:
a barrier defining an interior space for receiving the head of a patient and an opening through which the head may enter and leave the interior space, the barrier being arranged to prevent the transmission of aerosol particles through the barrier;
a suction port arranged to remove air from the interior space of the hood;
an access hole within the barrier, arranged to allow a person external to the barrier to access the interior space; and
an air jet arranged to propel air across the access hole in order to inhibit the transmission of aerosols through the access hole.

2. The hood of claim 1, wherein the hood comprises a plurality of air jets arranged to drive air across the access hole in order to inhibit the transmission of aerosols through the access hole.

3. The hood of claim 2, wherein the plurality of air jets are arranged parallel and are arranged in a row extending perpendicularly to a direction of flow of the jets.

4. The hood of claim 2, wherein the air jets are distributed around a boundary of the access hole and directed across the access hole.

5. The hood of claim 2, wherein the air jets are powered via a common manifold.

6. The hood of claim 2, wherein the air jets are powered by a common source.

7. The hood of claim 1, wherein the air jet is directed toward the suction port.

8. The hood of claim 1, wherein the air jet is a hole in the barrier arranged to permit the flow of air therethrough when a low pressure is created in the interior space by the removal of air via the suction port.

9. The hood of claim 1, further comprising a first flap extending at least partially across the access hole in a first direction.

10. The hood of claim 9, further comprising a second flap extending at least partially across the access hole in a second direction different from the first direction and arranged to overlap the first flap.

11. The hood of claim 10, wherein the first and second flaps have edges that are shaped as sinusoids in the region where the flaps overlap, and wherein the sinusoids are 180° out of phase.

12. The hood of claim 1, further comprising a first skirt extending from the barrier and arranged to at least partially obstruct the opening.

13. The hood of claim 12, wherein the first skirt has a quarter-spheroidal or quarter-ellipsoidal shape.

14. The hood of claim 1, further comprising a second skirt extending from the barrier and having an edge distal from the barrier, the edge having a concave shape.

15. The hood of claim 1, wherein the barrier is hemispheroidal or hemiellipsoidal.

16. An air flow system comprising:
the hood of claim 1; and
a suction pump coupled to the suction port and arranged to remove air from the interior space.

17. The air flow system of claim 16, further comprising: a jet pump coupled to one or more air jets and arranged to provide air to the one or more air jets at a pressure above atmospheric pressure.

18. The air flow system of claim 17, wherein the suction pump and the jet pump are the same pump.

19. The air flow system of claim 16, further comprising an air decontamination device arranged to sanitize the air passing through the air flow system.

20. The air flow system of claim 19, wherein the air decontamination device comprises an air filter or a UV sterilization device.
